(19) United States
(12) Patent Application Publication
(10) Pub. No.: US 2009/0239944 A1
(43) Pub. Date: Sep. 24, 2009

(54) Novel Use of Organic Compounds

(76) Inventors: Daniel D'orazio, Halten (CH); Antoine De Saizieu, Brunstatt (FR); Goede Schuler, Eimeldingen (DE); Ying Wang-Schmidt, Stallikon (CH); Christof Wehrli, Witterswil (CH); Swen Wolfram, Waldshut-Tiengen (DE)

Correspondence Address: NIXON & VANDERHYE, PC, 901 NORTH GLEBE ROAD, 11TH FLOOR, ARLINGTON, VA, 22203, US

(21) Appl. No.: 11/922,558

(22) PCT Filed: Jun. 23, 2006
(86) PCT No.: PCT/EP2006/006038

§ 371 Date: Dec. 20, 2007

(30) Foreign Application Priority Data
Jun. 24, 2005 (EP) 05 013 660.5

(51) Int. Cl. A61K 31/36; C07D 317/64; A61K 31/235; C07C 69/76

(52) U.S. Cl. 514/466; 549/438; 514/532; 560/108

(57) ABSTRACT

The present invention relates to compounds of the formula I

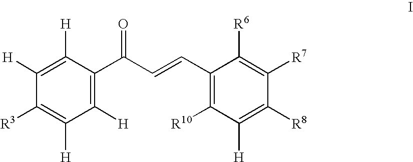

wherein R³ is C₁₋₆-alkyloxy, C₁₋₆-acyloxy or aroyloxy; R⁶ is hydrogen or C₁₋₆-alkyloxy; R⁷ is C₁₋₆-alkyloxy, C₁₋₆-acyloxy, aroyloxy or arylacyloxy; R⁸ is hydrogen or C₁₋₆-alkyloxy; or R⁷ and R⁸ form together a group O-L-O with L being (CR¹R²)ₙ, with R¹ and R² being independently from each other hydrogen or C₁₋₅-alkyl and n being an integer from 1 to 3; R¹⁰ is hydrogen or N—C₁₋₄-acyl, N—C₁₋₅-alkyl-x-Cₓ-alkyl with x being an integer from 1 to 5, for use as medicament for the treatment of a disorder connected to impaired glucose metabolism and impaired insulin action such as syndrome X and diabetes type 1 and 2, especially for the treatment of (non-autoimmune) diabetes type 2. The present invention is also directed to dietary compositions such as (fortified) food, beverages, (fortified) feed, food additives, beverage additives, feed additives, clinical nutrition, dietary supplements, functional food, functional feed and nutraceuticals and to pharmaceutical compositions containing such compounds, to methods of treating a disorder connected to impaired glucose metabolism and impaired insulin action in mammals including humans and to the compounds of the formula I themselves. Another object of the present invention is the use of such compounds for the manufacture of a composition for the treatment of a disorder connected to impaired glucose metabolism and impaired insulin action.

The present invention relates to compounds of the formula I

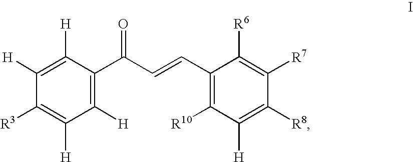

for use as medicament for the treatment of a disorder connected to impaired glucose metabolism and impaired insulin action. The present invention is also directed to dietary compositions such as (fortified) food, beverages, (fortified) feed, food additives, beverage additives, feed additives, clinical nutrition, dietary supplements, functional food, functional feed and nutraceuticals and to pharmaceutical compositions containing such compounds, to methods of treating a disorder connected to impaired glucose metabolism and impaired insulin action in mammals including humans and to the compounds of the formula I themselves. Another object of the present invention is the use of such compounds for the manufacture of a composition for the treatment of a disorder connected to impaired glucose metabolism and impaired insulin action.

The expression “treatment” hereby also encompasses co-treatment, control and prevention.

The expression “disorder” encompasses also diseases.

Such a disease connected to impaired glucose metabolism and impaired insulin action is diabetes mellitus, especially diabetes mellitus type 1 and 2, more especially (non-autoimmune) non-insulin dependent diabetes mellitus (NIDDM; so called Type 2 Diabetes). Another such disease is syndrome X.

Diabetes mellitus defines a complex of metabolic diseases derived from multiple causative factors and is characterized by impaired glucose metabolism, usually associated with impaired protein and fat metabolism. This results in elevated fasting and postprandial serum glucose that leads to complications if left untreated. Four different forms of diabetes mellitus are known, (1) type 1 diabetes mellitus, (2) type 2 diabetes mellitus, (3) the so-called gestational diabetes mellitus, which begins or is recognized for the first time during pregnancy, and (4) some other forms which are mainly based on genetic defects.

The term “diabetes mellitus” includes, but is not limited to, metabolic abnormalities such as increased blood glucose level, obesity associated pathologies, impaired glucose tolerance, increased insulin resistance, hyperlipidemia, dyslipidemia, increase in cholesterol (hypercholesterinemia, hypertriglycerinemia), hyperinsulinemia, hypertension, and microalbuminuria. Impaired glucose tolerance and impaired fasting glucose are the two symptoms referred to as pre-diabetes mellitus. This stage is associated with the so-called insulin resistance, one of a group of metabolic diseases called “syndrome X” or “metabolic syndrome”. Since type 2 diabetes mellitus is often associated with other symptoms from syndrome X, such as hypertriglyceridemia or dyslipidemia, the compounds according to the present invention are also useful for the treatment or prevention of syndrome X.

The two major forms of diabetes mellitus are the type 1 and type 2 diabetes mellitus, of which type 2 diabetes mellitus is the most prevailing form. Type 1 and type 2 diabetes mellitus are associated with hyperglycemia, hypercholesterolemia and hyperlipidemia. The insensitivity to insulin and absolute insulin deficiency in type 1 and 2 diabetes mellitus leads to a decrease in glucose utilization by the liver, muscle and the adipose tissue and to increased blood glucose levels. Uncontrolled hyperglycemia is associated with the dysfunction and failure of various organs such as the eyes, heart, blood vessels, kidney and nerves thus leading to increased and premature mortality due to an increased risk for microvascular and macrovascular diseases, including nephropathy, neuropathy, retinopathy, ulceration of the legs and feet, fatty liver disease, hypertension, cardiovascular diseases, and cerebrovascular diseases (stroke), the so-called diabetic complications. Recent evidence showed that tight glycemic control is a major factor in the prevention of these complications in both type 1 and type 2 diabetes mellitus. Therefore, optimal glycemic control by drugs or therapeutic regimens is an important approach for the treatment of diabetes mellitus.

Type 1 diabetes mellitus (autoimmune diabetes or insulin dependent diabetes mellitus (IDDM)) is the form of diabetes mellitus which usually begins with childhood or puberty and is characterized by an auto-immune destruction of the insulin-producing β-cells leading to a complete deficiency of insulin secretion. It develops due to an abnormal immune response against beta cells of pancreatic islets. The cause is complex and may involve genetics. Sometimes it follows a viral infection such as mumps, rubella, cytomegalovirus, measles, influenza, encephalitis or Epstein-Barr virus or environmental factors such as chemicals.

Type 2 diabetes mellitus (non-autoimmune diabetes or non-insulin dependent diabetes mellitus (NIDDM)) is the form of diabetes mellitus which occurs predominantly in adults in whom adequate production of insulin is available in the early stage of the diseases, yet a defect exists in insulin sensitivity, especially in insulin-mediated utilization and metabolism of glucose in peripheral tissues. Type 2 diabetes is a multi-factorial disorder that usually develops as a result of a sedentary lifestyle, a high caloric intake and excess body weight especially in those genetically predisposed. Thus, type 2 diabetes is often associated with insulin resistance and obesity rather than the lack of insulin like seen in type 1 diabetes. The changes in various tissues associated with type 2 diabetes mellitus exist even before clinical symptoms are detected.

Therapy of type 2 diabetes mellitus initially involves dietary and lifestyle changes. When these measures fail to maintain adequate glycemic control, the patients are treated with oral hypoglycemic agents and/or exogenous insulin. The current oral pharmacological agents for the treatment of type 2 diabetes mellitus include those that potentiate insulin secretion (sulphonylurea agents), those that improve the action of insulin in the liver (biguanide agents), insulin sensitizing agents (thiazolidinediones) and agents which act to inhibit the uptake of glucose in the gastrointestinal tract (α-glucosidase inhibitors). However, currently available agents generally fail to maintain adequate glycemic control in the long term due to progressive deterioration in hyperglycemia, resulting from progressive loss of pancreatic cell function. The proportion of patients able to maintain target glycemic levels decreases markedly overtime necessitating the administration of additional/alternative pharmacological agents. Furthermore, the drugs may have unwanted side effects and are associated with high primary and secondary failure rates.

Therefore, there is a need for compounds with minimal side effects for the prevention, control and/or treatment of disorders connected to impaired glucose metabolism and impaired insulin action, such as diabetes mellitus type 2 and Syndrome X, and for the prevention of the physical complications associated with it/them as mentioned above. Many patients are interested in alternative therapies which could minimize the side effects and drug resistance associated with high-dose of drugs and yield additive clinical benefits. In addition, people in high risk to develop Type 2 Diabetes, such as obese people, people with family history of Type 2 diabetes, and women with history of pregnancy diabetes, require early prevention measures. Type 2 diabetes mellitus is a progressive and chronic disease, which usually is not recognized until significant damage has occurred to the pancreatic cells responsible for producing insulin and to the cardiovascular system. Therefore, there is also an increasing interest in the development of a dietary supplement that may be used to prevent the development of diabetes mellitus in people at risk especially in elderly persons, but also in obese children, who are at high risk for developing diabetes mellitus.

We now found that compounds of the formula I

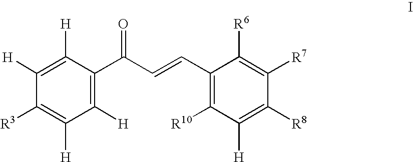

wherein R³ is C₁₋₆-alkyloxy, C₁₋₆-acyloxy or aroyloxy;
R⁶ is hydrogen or C₁₋₆-alkyloxy;
R⁷ is C₁₋₆-alkyloxy, C₁₋₆-acyloxy, aroyloxy or arylacyloxy;
R⁸ is hydrogen or C₁₋₆-alkyloxy;
or R⁷ and R⁸ form together a group O-L-O with L being (CR¹R²)ₙ, with R¹ and R² being independently from each other hydrogen or C₁₋₅-alkyl and n being an integer from 1 to 3;
R¹⁰ is hydrogen or N—C₁₋₄-acyl, N—C₁₋₅-alkyl-x-Cₓ-alkyl with x being an integer from 1 to 5, preferably with the proviso that R³ is not methoxy when R⁶ and R¹⁰ are hydrogen and R⁷ and R⁸ are methoxy, may be effective agents in the prevention, control and/or treatment of disorders connected to impaired glucose metabolism and impaired insulin action in mammals including humans such as diabetes mellitus type 2 and syndrome X, especially (non auto-immune) type 2 diabetes and non-autoimmune beta cell dysfunction.

Therapeutic effects of these compounds may include, but are not limited to, the following ones. Therefore, the present invention is directed to the use of the compounds of the formula I as defined above for
helping to manage blood sugar levels, i.e. helping the body by balancing the blood sugar levels; helping to keep balanced blood glucose levels, particularly in humans with diabetes; aiding by enhancing the glucose uptake by the cells and by reducing sugar levels, thus improving or restoring the glucose tolerance; lowering the blood glucose level; optimizing the glycemic response; normalizing the glucose tolerance; i.e. the compounds of the formula I may be α-glucosidase inhibitors, hyperglycemia treating and/or controlling agents and blood glucose controlling agents;
reducing sweetness cravings;
preserving or improving the pancreatic β-cell function, thus promoting a healthy pancreatic function; i.e. the compounds of the formula I may be pancreatic β-cell function improvers;
treating or controlling the insulin resistance/sensitivity by e.g. helping to restore/enhance the insulin sensitivity in peripheral tissues, such as adipose, liver and skeletal muscle; i.e. the compounds of the formula I may be insulin sensitizers;
lowering insulin resistance;
delaying, preventing or controlling diabetes mellitus type 2, especially NIDDM, and dyslipidemia and thus preventing also the diabetes accompanying disorders/complications such as the ones mentioned above; i.e. the compounds of the formula I are diabetes type 2 preventing agents;
activating adipocytes, thus increasing insulin sensitivity;
repartioning of fat from lipolytic visceral fat depots into subcutaneous fat depots, thus decreasing the risk of obesity associated pathologies such as cardiovascular diseases;
reducing the circulation of free fatty acids (FFA), thus improving the insulin sensitivity in obese people;
maintaining endothelial function;
lowering triglyceride levels in the blood; maintaining a healthy/normal blood lipid balance and a healthy/normal blood lipid profile by regulating/adjusting the blood lipid levels thus optimizing the blood lipid profile; treating elevated blood lipid levels and high blood cholesterol levels by metabolizing cholesterol and blood lipids; helping to reduce the cholesterol levels in a hyperlipidemic patient; improving dyslipidemia; i.e. the compounds of the formula I may be blood lipids lowering agents.

The compounds of the present invention are particularly intended for the treatment and control of both type 1 and 2 diabetes, and for the prevention of type 2 diabetes in those individuals in high risk to develop this disease, such as individuals with pre-diabetes, impaired glucose tolerance (IGT), or obesity.

Therefore, the present invention is directed to compounds of the formula I,

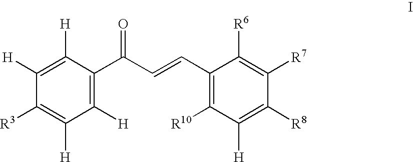

wherein R³ is C₁₋₆-alkyloxy, C₁₋₆-acyloxy or aroyloxy;
R⁶ is hydrogen or C₁₋₆-alkyloxy;
R⁷ is C₁₋₆-alkyloxy, C₁₋₆-acyloxy, aroyloxy or arylacyloxy;
R⁸ is hydrogen or C₁₋₆-alkyloxy;
or R⁷ and R⁸ form together a group O-L-O with L being (CR¹R²), with R¹ and R² being independently from each other hydrogen or C₁₋₅-alkyl and n being an integer from 1 to 3;
R¹⁰ is hydrogen or N—C₁₋₄-acyl, N—C₁₋₅-alkyl-x-Cₓ-alkyl with x being an integer from 1 to 5; preferably with the proviso that R³ is not methoxy when R⁶ and R¹⁰ are hydrogen and R⁷ and R⁸ are methoxy, for use as medicament for the treatment of a disorder connected to impaired glucose metabolism and impaired insulin action.

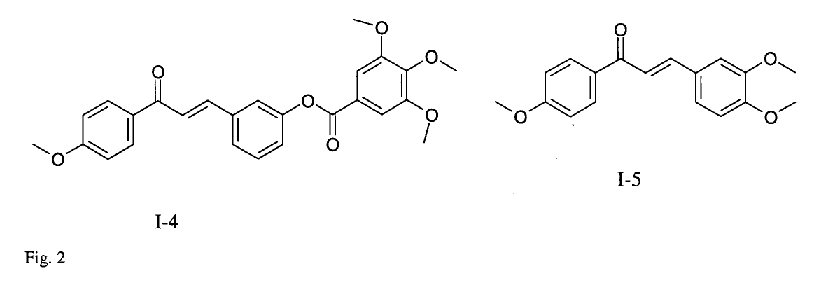

Especially preferred for such use are compounds of the formula I, wherein
R³ is methoxy, isopropyloxy, isoprenyloxy, acetyloxy, or benzoyloxy, especially wherein R³ is methoxy or benzoyloxy; and/or
R⁶ is hydrogen, methoxy, isopropyloxy or isoprenyloxy, especially wherein R⁶ is hydrogen or methoxy; and/or
R⁷ is methoxy, isopropyloxy, isoprenyloxy, acetyloxy, benzoyloxy, (3,4,5-trimethoxy)benzoyloxy or cinnamoyloxy, especially wherein R⁷ is methoxy or cinnamoyloxy; and/or
R⁸ is hydrogen, methoxy, isopropyloxy or isoprenyloxy, especially wherein R⁸ is hydrogen or methoxy;
   or
R⁷ and R⁸ form together the group O(—CH₂)ₘ—O with m being 1 or 2; and/or
R¹⁰ is hydrogen, N-acetyl, N-isopropyl-2-aminoethyl, N-isoprenyl-2-aminoethyl or N-acetyl, N-methyl-2-aminoethyl, especially wherein R¹⁰ is hydrogen or N-acetyl, N-methyl-2-aminoethyl,
   with the proviso that R³ is not methoxy when R⁶ and R¹⁰ are hydrogen and R⁷ and R⁸ are methoxy (compound of the formula I-5 as shown in FIG. 2).

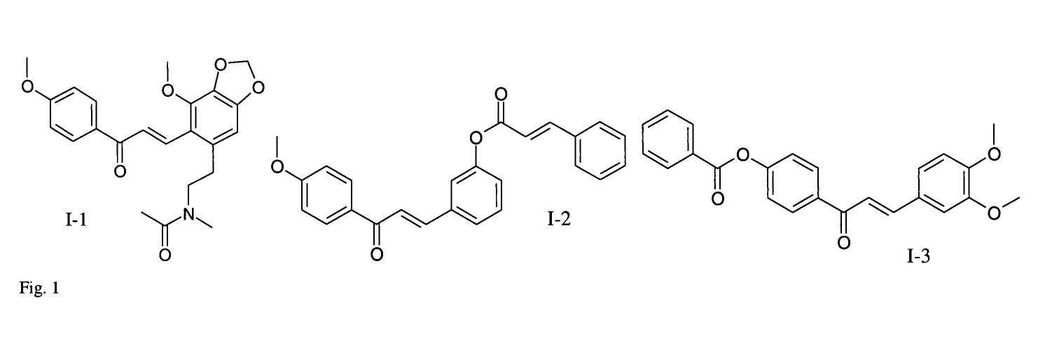

In an especially preferred embodiment of the present invention a compound selected from the group consisting of
the compound of the formula I, in which R³ and R⁶ are both methoxy (OCH₃), R⁷ and R⁸ form together a group O—CH₂—O and R¹⁰ is N-acetyl, N-methyl-2-aminoethyl (=compound of the formula I-1);
the compound of the formula I, in which R³ is OCH₃, R⁶=R⁸=R¹⁰=hydrogen, and R⁷=cinnamoyloxy (=compound of the formula I-2); and
the compound of the formula I, in which R³=benzoyloxy, R⁶=R¹⁰=hydrogen, and R⁷=R⁸=OCH₃ (=compound of the formula I-3);
the compound of the formula I, in which R³=OCH₃, R⁶=R⁸=R¹⁰=hydrogen, and R⁷=(3,4,5-trimethoxy)benzoyloxy (=compound of the formula I-4);
   or mixtures thereof are used. Even more preferred are the compounds of the formulae I-1, I-2 and I-3. The formulae of the compounds I-1 to I-3 are shown in FIG. 1, the formula of the compound I-4 is shown in FIG. 2.

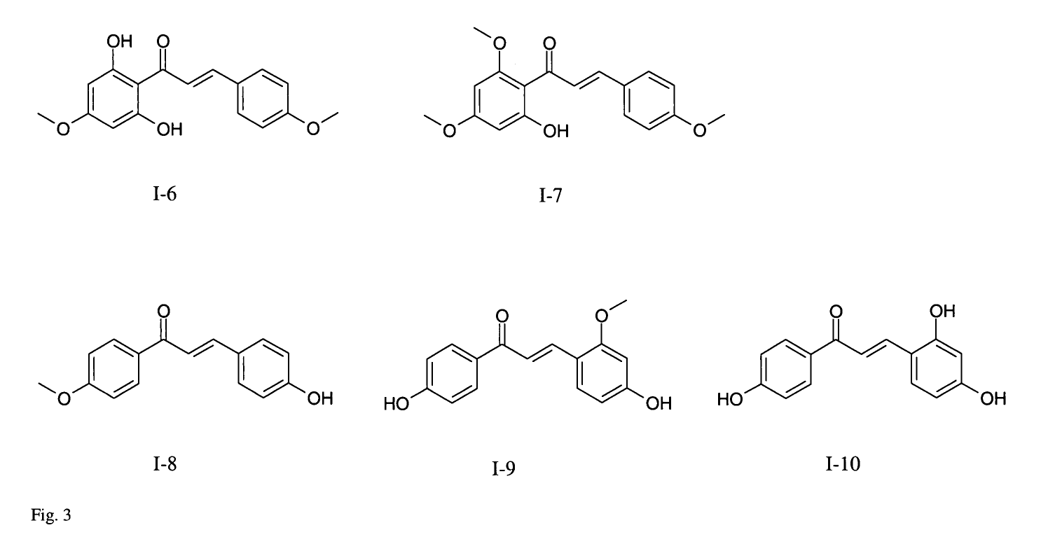

Interestingly compounds of formula I-6 to I-10 (FIG. 3) are not active in the tests disclosed in the examples.

The term “compound of the formula I” also encompasses any material or extract of a plant containing such a compound of the formula I, preferably in an amount of at least 30 weight-% (i.e. in an amount of from 30 to 100 weight-%), more preferably in an amount of at least 50 weight-% (i.e. in an amount of from 50 to 100 weight-%), even more preferably in an amount of at least 70 weight-% (i.e. in an amount of from 70 to 100 weight-%), most preferably in an amount of at least 90 weight-% (i.e. in an amount of from 90 to 100 weight-%), based on the total weight of the plant material or extract. The terms “material of a plant” and “plant material” used in the context of the present invention mean any part of a plant.

The compound of the formula I-1 can be isolated from plants like Papaver pseudo orientate and the poppy plant, but not limited to them.

Therefore, any material or extract of these plants or any other plant material or extract containing the compound of the formula I-1, preferably in an amount of at least 30 weight-%, more preferably in an amount of at least 50 weight-%, even more preferably in an amount of at least 70 weight-%, most preferably in an amount of at least 90 weight-%, based on the total weight of the plant material or extract, is also encompassed by this expression. “Compound of the formula I-1” means both “natural” (isolated) and “synthetic” (manufactured) compound of the formula I-1.

The compound of the formula I-2 can be isolated from plants like Glycyrrhiza glabra (licorice), but not limited to it.

Therefore, any material or extract of these plants or any other plant material or extract containing the compound of the formula I-2, preferably in an amount of at least 30 weight-%, more preferably in an amount of at least 50 weight-%, even more preferably in an amount of at least 70 weight-%, most preferably in an amount of at least 90 weight-%, based on the total weight of the plant material or extract, is also encompassed by this expression. “Compound of the formula I-2” means both “natural” (isolated) and “synthetic” (manufactured) compound of the formula I-2.

The compound of the formula I-3 can be isolated from plants like Glycyrrhiza glabra (licorice) and the poppy plant, but not limited to them.

Therefore, any material or extract of these plants or any other plant material or extract containing the compound of the formula I-3, preferably in an amount of at least 30 weight-%, more preferably in an amount of at least 50 weight-%, even more preferably in an amount of at least 70 weight-%, most preferably in an amount of at least 90 weight-%, based on the total weight of the plant material or extract, is also encompassed by this expression. “Compound of the formula I-3” means both “natural” (isolated) and “synthetic” (manufactured) compound of the formula I-3.

The compound of the formula I-4 can be isolated from plants like Glycyrrhiza glabra (licorice), but not limited to it.

Therefore, any material or extract of these plants or any other plant material or extract containing the compound of the formula I-4, preferably in an amount of at least 30 weight-%, more preferably in an amount of at least 50 weight-%, even more preferably in an amount of at least 70 weight-%, most preferably in an amount of at least 90 weight-%, based on the total weight of the plant material or extract, is also encompassed by this expression. “Compound of the formula I-4” means both “natural” (isolated) and “synthetic” (manufactured) compound of the formula I-4.

Beside the (pure) compounds of the formula I-1, I-2, I-3 and I-4 preferred are plant materials and plant extracts, especially those containing at least 30 weight-%, preferably at least 50 weight-%, more preferably at least 70 weight-%, most preferably at least 90 weight-%, of these compounds, based on the total weight of the plant material/extract.

The present invention is further directed to the use of a compound of the formula I as defined above for the manufacture of a composition for the treatment of a disorder connected to impaired glucose metabolism and impaired insulin action.

In preferred embodiments of the present invention this composition is used as blood glucose controlling agent, as insulin sensitizer, as blood lipid lowering agent, as pancreatic β-cell function improver, as diabetes type 2 preventing agent and/or as Syndrome X preventing agent.

The present invention is also directed to a dietary composition containing at least a compound of the formula I

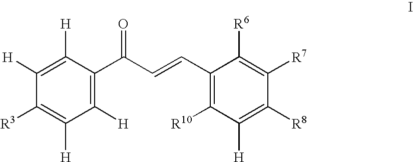

wherein R³ is C₁₋₄-alkyloxy, C₁₋₆-acyloxy or aroyloxy;
R⁶ is hydrogen or C₁₋₆-alkyloxy;
R⁷ is C₁₋₆-alkyloxy, C₁₋₆-acyloxy, aroyloxy or arylacyloxy;
R⁸ is hydrogen or C₁₋₆-alkyloxy;
or R⁷ and R⁸ form together a group O-L-O with L being (CR¹R²)ₙ, with R¹ and R² being independently from each other hydrogen or C₁₋₅-alkyl and n being an integer from 1 to 3;
R¹⁰ is hydrogen or N—C₁₋₄-acyl, N—C₁₋₅-alkyl-x-Cₓ-alkyl with x being an integer from 1 to 5, preferably with the proviso that R³ is not methoxy when R⁶ and R¹⁰ are hydrogen and R⁷ and R⁸ are methoxy (i.e. preferably the compound of formula I is not the compound of the formula I-5 as shown in FIG. 2).

R³ is preferably methoxy, isopropyloxy, isoprenyloxy, acetyloxy, or benzoyloxy, more preferably R³ is methoxy or benzoyloxy.

R⁶ is preferably hydrogen, methoxy, isopropyloxy or isoprenyloxy, more preferably R⁶ is hydrogen or methoxy.

R⁷ is preferably methoxy, isopropyloxy, isoprenyloxy, acetyloxy, benzoyloxy, (3,4,5-trimethoxy)benzoyloxy or cinnamoyloxy, more preferably R⁷ is methoxy or cinnamoyloxy.

R⁸ is preferably hydrogen, methoxy, isopropyloxy or isoprenyloxy, more preferably R⁸ is hydrogen or methoxy.

Preferred is also a dietary composition containing a compound of the formula I, wherein R⁷ and R⁸ form together the group O(CH₂)ₘ—O with m being 1 or 2, especially wherein R⁷ and R⁸ form together the group O—CH₂—O.

R¹⁰ is preferably hydrogen, N-acetyl, N-isopropyl-2-aminoethyl, N-isoprenyl-2-aminoethyl or N-acetyl, N-methyl-2-aminoethyl, more preferably R¹⁰ is hydrogen or N-acetyl, N-methyl-2-aminoethyl.

In a preferred embodiment of the invention the dietary composition contains at least a compound selected from the group consisting of compounds of the formula I-1 to I-4, especially compounds of the formula I-1 to I-3, as defined above.

The term “dietary compositions” comprises any type of (fortified) food, (fortified) (animal) feed and beverages including also clinical nutrition, and also dietary supplements as well as the corresponding additives: food additives, beverage additives, feed additives. Also encompassed is functional food/feed i.e. a food/feed that has been enhanced with vitamins, other micronutrients or pharmaceuticals to provide further specific health benefits, as well as a nutraceutical, i.e. a pill or other pharmaceutical product that has nutritional value.

The dietary compositions according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellyfying agents, gel forming agents, antioxidants and antimicrobials.

A pharmaceutical composition containing at least one compound of the formula I with the definitions of R¹ to R¹⁰ and the preferences as given above and a conventional pharmaceutical carrier.

Especially preferred is a pharmaceutical composition wherein the compound of the formula I is selected from the group consisting of compounds of the formula I-1 to I-4, especially compounds of the formula I-1 to I-3, as defined above.

Beside a pharmaceutically acceptable carrier and at least one compound of the formula I with the definitions of R¹ to R¹⁰ and the preferences as given above, the pharmaceutical compositions according to the present invention may further contain conventional pharmaceutical additives and adjuvants, excipients or diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. The carrier material can be organic or inorganic inert carrier material suitable for oral/parenteral/injectable administration.

The dietary and pharmaceutical compositions according to the present invention may be in any galenic form that is suitable for administrating to the animal body including the human body, especially in any form that is conventional for oral administration, e.g. in solid form such as (additives/supplements for) food or feed, food or feed premix, fortified food or feed, tablets, pills, granules, dragées, capsules, and effervescent formulations such as powders and tablets, or in liquid form such as solutions, emulsions or suspensions as e.g. beverages, pastes and oily suspensions. The pastes may be filled into hard or soft shell capsules, whereby the capsules feature e.g. a matrix of (fish, swine, poultry, cow) gelatin, plant proteins or ligninsulfonate. Examples for other application forms are forms for transdermal, parenteral or injectable administration. The dietary and pharmaceutical compositions may be in the form of controlled (delayed) release formulations.

Examples for fortified food are cereal bars, bakery items such as cakes and cookies.

Beverages encompass non-alcoholic and alcoholic drinks as well as liquid preparations to be added to drinking water and liquid food. Non-alcoholic drinks are e.g. soft drinks, sport drinks, fruit juices, lemonades, teas and milk based drinks. Liquid food are e.g. soups and dairy products.

The compounds of the formula I with the definitions of R¹ to R¹⁰ and the preferences as given above as well as (mixtures of) plant materials and plant extracts containing them, preferably in an amount of at least 30 weight-%, more preferably in an amount of at least 50 weight-%, even more preferably in an amount of at least 70 weight-%, most preferably in an amount of at least 90 weight-%, based on the total weight of the plant material or extract, and dietary/pharmaceutical compositions containing them are thus suitable for the treatment of mammals including humans.

Therefore, the invention relates to a method for the treatment of a disorder connected to impaired glucose metabolism and impaired insulin action in mammals including humans, said method comprising administering an effective dose of a compound of the formula I as defined above to mammals including humans which are in need thereof.

Mammals in the context of the present invention include humans. Preferred “mammals” are humans, and pets such as cats, dogs and horses, especially dogs.

In the context of this invention “treatment” also encompasses co-treatment as well as control and or prevention. In the context of this invention the term “disorder” also encompasses diseases.

For humans a suitable daily dosage of a compound of the formula I with the definitions of R¹ to R¹⁰ and the preferences as given above, for the purposes of the present invention may be within the range from 0.01 mg per kg body weight to 50 mg per kg body weight per day.

More preferred is a daily dosage of 0.1 to 25 mg per kg body weight, and especially preferred is a daily dosage of 0.3 to 15 mg per kg body weight. The amount of a plant material or plant extract containing such compound of the formula I can be calculated accordingly.

In solid dosage unit preparations for humans, the compound of the formula I with the definitions of R¹ to R¹⁰ and the preferences as given above is suitably present in an amount from 0.25 mg to 1000 mg, preferably from 2 mg to 200 mg per dosage unit.

In dietary compositions, especially in food and beverages for humans, the compound of the formula I with the definitions of R¹ to R¹⁰ and the preferences as given above may suitably be present in an amount of from 0.5 mg/kg to 100 g/kg, preferably from 5 mg/kg to 10 g/kg, more preferably from 50 mg/kg to 2 g/kg, based upon the total weight of the food or beverage.

In food and drinks in a preferred embodiment of the invention the amount of the compound of the formula I with the definitions of R¹ to R¹⁰ and the preferences as given above may be 0.7 to 4000 mg per serving.

For dogs a suitable daily dosage of a compound of the formula I with the definitions of R¹ to R¹⁰ and the preferences as given above for the purposes of the present invention may be within the range from 0.04 mg per kg body weight to 500 mg per kg body weight per day. More preferred is a daily dosage of 0.4 mg to 100 mg per kg body weight, and especially preferred is a daily dosage of 1 mg to 50 mg per kg body weight.

The present invention is also directed to compounds of the formula I, as defined above, especially to compounds of the formula I

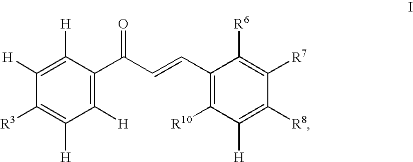

wherein R³ is methoxy or benzoyloxy,
R⁶ is hydrogen or methoxy,
R⁷ is methoxy or cinnamoyloxy or (3,4,5-trimethoxy)benzoyloxy,
R⁸ is hydrogen or methoxy,
or R⁷ and R⁸ form together the group O(—CH₂)ₘ—O with m being 1 or 2, and
R¹⁰ is hydrogen or N-acetyl, N-methyl-2-aminoethyl; with the proviso that R³ is not methoxy when R⁶ and R¹⁰ are hydrogen and R⁷ and R⁸ are methoxy,
especially to the compound of the formula I, wherein R³ and R⁶ are both OCH₃, R⁷ and R⁸ form together a group O—CH₂—O and R¹⁰ is N-acetyl, N-methyl-2-aminoethyl (=compound of the formula I-1), as well as to their use as medicament.

The invention is now further illustrated by the following examples.

### EXAMPLES

The following abbreviations are used:

BW=body weight
DMEM=Dulbecco's Modified eagle Medium
DMSO=dimethylsulfoxide
FBS=Fetal Bovine serum
2-DG=2-deoxyglucose
3-[H]-2-DG=tritiated 2-deoxyglucose
HBS=Hanks balanced salt solution

### oil Red O=Solvent Red 27 (CAS-No. 1320-06-5)

PBS=Phosphate buffer solution
OD=optical density
SEM=standard error of the mean
FFA=free fatty acids

### GUA=Glucose Uptake of Adipocytes

### Example 1

### Effect of the Compound of the Formula I-1 on the Glucose Uptake of Adipocytes

C3H10T1/2 cells (ATCC CCL-226) were grown for 5 days to confluence in DMEM supplemented with 10% FBS medium and induced with a mixture of insulin, dexamethasone and 3-isobutyl-1-methylxanthine to differentiate into adipocytes. Nine days after the beginning of induction, cells were treated for 48-h with the compound of the formula I-1 at different concentrations as shown in Table 1. Glucose uptake was determined using radioactive 2-deoxyglucose (10 μM 2-DG in HBS+0.5 μCi/ml of 3-[H]-2-DG), measuring glucose uptake in the absence of insulin. Basal glucose uptake was increased by 48-h treatment with the compound of the formula I-1 in a dose-dependent manner (Table 1). As a positive control, the known PPARγ agonist ciglitazone was used in the concentration as indicated in Table 1.

### Example 2

### Effect of the Compound of the formula I-3 on Glucose Uptake of Adipocytes

Growing, induction and treatment of C3H10T1/2 cells were exactly as described in Example 1, with the exception that the compound of the formula I-3 at different concentrations was used instead of the compound of the formula I-1. As shown in Table 1, an increase of the basal glucose uptake could be detected.

TABLE 1
Induction of glucose uptake by 48-h treatment with different
compounds (% of control ± SEM)

| | Concentration | |
|---|---|---|
| Compound | [M] | Basal glucose uptake |
| Ciglitazone | 5 × 10⁻⁵ | 496.178 ± 61.86 |
| compound of the formula I-1 | 1 × 10⁻⁶ | 97.6 ± 22.9 |
| | 1 × 10⁻⁵ | 94.58 ± 0.95 |
| | 2.4 × 10⁻⁵ | 128.6 ± 0.89 |
| | 5 × 10⁻⁴ | 194.058 ± 0.05 |
| compound of the formula I-3 | 1 × 10⁻⁶ | 105.37 ± 2.21 |
| | 1 × 10⁻⁵ | 133.28 ± 23.4 |
| | 5 × 10⁻⁵ | 116.17 ± 15.96 |
| | 2 × 10⁻⁴ | 142.84 ± 18.05 |

Control: C3H10T1/2 cells treated for 48 h with DMSO at the same concentration as compound-treated cells and set at 100%

### Example 3

### Effect of the Compound of the Formula I-1 on Differentiation of Adipocytes

C3H10T1/2 cells were grown to confluence as described in Example 1, then treated for 10 days with insulin alone (negative control) or with a mixture of insulin and the compound of the formula I-1 at different concentrations (see Table 2), with re-feeding with fresh medium and compounds every 48-h. After 10-days of treatment, the cells were stained with oil Red O as follows: cells were washed 2× in PBS and fixed in 10% formalin at room temperature for 1 h. After removal of formalin, 200 μl of oil Red O staining solution (3:2 mixture of 0.5% w/v oil Red O stock solution and water) was applied to each well. The cells were incubated for 20 min at room temperature, washed twice in 2×PBS and incubated for 10 min with 300 μl of isopropanol/well for oil Red O extraction. Quantification of oil Red O was determined by measuring absorbance at 540 nm (mean OD). Co-treatment of C3H10T1/2 cells with insulin and the compound of the formula I-1 resulted in a higher differentiation of the cells into adipocytes than insulin alone as represented by a higher amount of oil Red O staining (Table 2).

TABLE 2
Induction of adipocyte differentiation by 10-day treatment with the
compound of the formula I-1

| Compound | Mean OD ± SEM |
|---|---|
| Insulin (1 × 10⁻⁷ M) | 0.28 ± 0.03 |
| Insulin (1 × 10⁻⁷ M) + compound of the formula I-1 | 0.69 ± 0.019 |
| (1 × 10⁻⁵ M) | |

### Example 4

### Effect of the Compound of the Formula I-2 on Differentiation of Adipocytes

C3H10T1/2 cells were grown and treated as described in Example 4 with the exception that the compound of the formula I-2 was used instead of the compound of the formula I-1. The measurement of adipocyte differentiation using the oil Red O assay was performed as described in Example 4. Co-treatment of C3H10T1/2 cells with insulin and the compound of the formula I-2 resulted in a higher differentiation of the cells into adipocytes than insulin alone (Table 3).

### Example 5

### Effect of Compound of the Formula I-3 on Differentiation of Adipocytes

C3H10T1/2 cells were grown and treated as described in Example 4 with the exception that the compound of the formula I-3 was used instead of the compound of the formula I-1. The measurement of adipocyte differentiation using the oil Red O assay was performed as described in Example 4. Co-treatment of C3H10T1/2 cells with insulin and the compound of the formula I-3 resulted in a higher differentiation of the cells into adipocytes than insulin alone (Table 3).

TABLE 3
Induction of adipocyte differentiation by 10-day treatment with the
compound of the formula I-2 or the compound of the formula I-3.

| Compound | Mean OD ± SEM |
|---|---|
| Insulin (1 × 10⁻⁷ M) | 0.28 ± 0.030 |
| Insulin (1 × 10⁻⁷ M) + compound of the formula I-2 | 0.45 ± 0.037 |
| (1 × 10⁻⁵ M) | |
| Insulin (1 × 10⁻⁷ M) + compound of the formula I-3 | 0.53 ± 0.025 |
| (1 × 10⁻⁵ M) | |
| Insulin (1 × 10⁻⁷ M) + compound of the formula I-3 | 0.53 ± 0.017 |
| (5 × 10⁻⁵ M) | |
| Insulin (1 × 10⁻⁷ M) + compound of the formula I-3 | 0.34 ± 0.087 |
| (2 × 10⁻⁴ M) | |

### Example 6

### Effect of the Compound of the Formula I-1 on Glucose Tolerance

The efficacy of the compound of the formula I-1 on glucose tolerance was tested in a 14-day study in C57BLKS/J db/db mice (n=10/group), a model of late type 2 diabetes mellitus with severe hyperglycemia.

Male db/db mice were obtained from Jackson Laboratory (Bar Harbor, Me., USA). Adult mice aged 8 weeks were used in the experiment. Mice were housed individually in plastic cages with bedding and allowed free access to standard rodent food and tap water. The animal rooms were controlled for temperature (24° C.), humidity (55%), and light (12-h light-dark cycle). The animals were randomized in two groups and the compound of the formula I-1 was administered orally to one of the groups for 14 days at a dose of 200 mg/kg BW/day. After 14 days of treatment the concentration of glucose was determined in blood from fed animals, i.e., animals which were not restricted from food. After a period of 10 days of treatment an oral glucose tolerance test (OGTT) was performed. For the OGTT mice were fasted overnight and then a 1-g glucose/kg BW solution was orally administered. Blood samples were taken before and 15, 30, 45, 60, 90, 120, 150, 180 min after the glucose challenge for determination of blood glucose levels and then the area under the curve (AUC) was determined. Blood glucose was measured by a glucose analyzer (Glucotrend Premium, Roche Diagnostics, Rotkreuz, Switzerland). The blood glucose levels and AUC for the OGTT measurement are given in Table 4. The glucose and the free fatty acid (FFA) levels of fed animals (see above) were lowered after 14 days of treatment with the compound of the formula I-1. After 10 days of treatment with the compound of the formula I-1 the glucose levels of fasted animals, i.e., animals with an overnight fasting (see above) were decreased as compared to the untreated control group. During the OGTT test the blood glucose levels in the animals treated with the compound of the formula I-1 were lower at all time points when compared with the control group. Thus, the compound of the formula I-1 significantly reduced the glucose AUC of an OGTT (1 g glucose/kg body weight) on day 10.

TABLE 4
Blood glucose level in db/db mice treated with the compound
of the formula I-1

| | Blood Glucose | | | |
|---|---|---|---|---|
| | Fasted | Fed | Glucose | |
| | (mg/dl) | (mg/dl) | AUC | FFA (mg/dl) |
| Control | 175 | 859 | 69673 | 15.95 |
| compound of the formula I-1 | 135 | 771 | 54295 | 12.38 |
| (200 mg/kg BW/day) | | | | |

1. A dietary composition containing at least a compound of the formula I 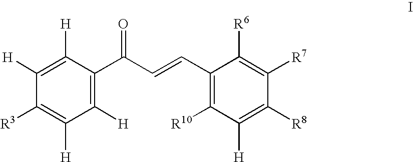
wherein R³ is C₁₋₆-alkyloxy, C₁₋₆-acyloxy or aroyloxy;
R⁶ is hydrogen or C₁₋₆-alkyloxy;
R⁷ is C₁₋₆-alkyloxy, C₁₋₆-acyloxy, aroyloxy or arylacyloxy;
R⁸ is hydrogen or C₁₋₆-alkyloxy;
or R⁷ and R⁸ form together a group O-L-O with L being (CR¹R²)ₙ, with R¹ and R² being independently from each other hydrogen or C₁₋₅-alkyl and n being an integer from 1 to 3;
R¹⁰ is hydrogen or N—C₁₋₄-acyl, N—C₁₋₅-alkyl-x-Cₓ-alkyl with x being an integer from 1 to 5,
preferably with the proviso that the composition does not contain a compound of formula I wherein R³ is methoxy when R⁶ and R¹⁰ are hydrogen and R⁷ and R⁸ are methoxy.

2. The dietary composition according to claim 1, wherein
R³ is methoxy, isopropyloxy, isoprenyloxy, acetyloxy, or benzoyloxy, preferably wherein R³ is methoxy or benzoyloxy; and/or
wherein R⁶ is hydrogen, methoxy, isopropyloxy or isoprenyloxy, preferably wherein R⁶ is hydrogen or methoxy; and/or
wherein R⁷ is methoxy, isopropyloxy, isoprenyloxy, acetyloxy, benzoyloxy, (3,4,5-trimethoxy)benzoyloxy or cinnamoyloxy, preferably wherein R⁷ is methoxy or cinnamoyloxy; and/or
wherein R⁸ is hydrogen, methoxy, isopropyloxy or isoprenyloxy, preferably wherein R⁸ is hydrogen or methoxy; or
wherein R⁷ and R⁸ form together the group O(—CH₂)ₘ—O with m being 1 or 2, preferably wherein R⁷ and R⁸ form together the group 0-CH₂—O; and/or
wherein R¹⁰ is hydrogen, N-acetyl, N-isopropyl-2-aminoethyl, N-isoprenyl-2-aminoethyl or N-acetyl, N-methyl-2-aminoethyl, preferably wherein R¹⁰ is hydrogen or N-acetyl, N-methyl-2-aminoethyl, preferably with the proviso that R³ is not methoxy when R⁶ and R¹⁰ are hydrogen and R⁷ and R⁸ are methoxy.

3. The dietary composition according to claim 1, wherein the compound of the formula I is selected from the group consisting of the compounds of the formula I-1 to I-3 wherein the compound of the formula I-1 is one, in which R³=R⁶=OCH₃, R⁷ and R⁸ form together a group O—CH₂—O and R¹⁰=N-acetyl, N-methyl-2-aminoethyl; the compound of the formula I-2 is one, in which R³=OCH₃, R⁶=R⁸=R¹⁰=H, and R⁷=cinnamoyloxy; the compound of the formula I-3 is one, in which R³=benzoyloxy, R⁶=R¹⁰=H, and R⁷=R⁸=OCH₃; and the compound of the formula I-4 is one, in which R³=OCH₃, R⁶=R⁸=R¹⁰=H, and R⁷=(3,4,5-trimethoxy)benzoyloxy.

4. The dietary composition as in claim 1, wherein the dietary composition is (fortified) food, a beverage, (fortified) feed or a corresponding additive, functional food, functional feed, nutraceutical, clinical nutrition or a dietary supplement.

5. A compound of the formula I as defined in claim 1 for the manufacture of a composition for the treatment of a disorder connected to impaired glucose metabolism and impaired insulin action.

6. The composition according to claim 5, wherein the composition is used as a blood glucose controlling agent, as an insulin sensitizer, as a blood lipid lowering agent, as a pancreatic β-cell function improver, as a diabetes type 2 preventing agent and/or as a Syndrome X preventing agent.

7. A disorder according to claim 5, wherein the disorder connected to impaired glucose metabolism and impaired insulin action is diabetes mellitus or syndrome X, especially non-autoimmune diabetes type 2.

8. Compounds of the formula I, 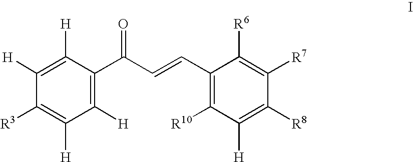
wherein R³ is methoxy or benzoyloxy,
R⁶ is hydrogen or methoxy,
R⁷ is methoxy or (3,4,5-trimethoxy)benzoyloxy or cinnamoyloxy,
R⁸ is hydrogen or methoxy,
or R⁷ and R⁸ form together the group O(—CH₂)ₘ—O with m being 1 or 2, and
R¹⁰ is hydrogen or N-acetyl, N-methyl-2-aminoethyl, with the proviso that R³ is not methoxy when R⁶ and R¹⁰ are hydrogen and R⁷ and R⁸ are methoxy.

9. The compound of the formula I according to claim 8, wherein R³ and R⁶ are both OCH₃, R⁷ and R⁸ form together a group O—CH₂—O and R¹⁰ is N-acetyl, N-methyl-2-aminoethyl.

10. Compounds of the formula I, 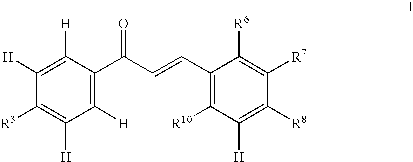
wherein R³ is C₁₋₆-acyloxy or aroyloxy,
R⁶ is hydrogen or d-₆-alkyloxy,
R⁷ is C₁₋₆-alkyloxy, Ci.₆-acyloxy, aroyloxy or arylacyloxy,
R⁸ is hydrogen or C₁₋₆-alkyloxy,
or R⁷ and R⁸ form together a group O-L-O with L being (CR¹R²)ₙ, with R¹ and R² being independently from each other hydrogen or C₁₋₅-alkyl and n being an integer from 1 to 3;
R¹⁰ is hydrogen or N—C₁₋₄-acyl, N—C₁₋₅-alkyl-x-Cₓ-alkyl with x being an integer from 1 to 5; preferably with the proviso that R³ is not methoxy when R⁶ and R¹⁰ are hydrogen and R⁷ and R⁸ are methoxy,
for use as medicament for the treatment of a disorder connected to impaired glucose metabolism and impaired insulin action.

11. Compound of the formula I according to claim 10, wherein R³ is methoxy, isopropy-loxy, isoprenyloxy, acetyloxy, or benzoyloxy, especially wherein R³ is methoxy or benzoyloxy.

12. Compound of the formula I according to claim 10, wherein R⁶ is hydrogen, methoxy, isopropyloxy or isoprenyloxy, especially wherein R⁶ is hydrogen or methoxy.

13. Compound of the formula I according to claim 10, wherein R⁷ is methoxy, isopropyloxy, isoprenyloxy, acetyloxy, benzoyloxy, (3,4,5-trimethoxy)benzoyloxy or cinnamoyloxy, especially wherein R⁷ is methoxy or cinnamoyloxy.

14. Compound of the formula I according to claim 10, wherein R⁸ is hydrogen, methoxy, isopropyloxy or isoprenyloxy, especially wherein R⁸ is hydrogen or methoxy.

15. Compound of the formula I according to claim 10, wherein R⁷ and R⁸ form together the group O(—CH₂)ₘ—O with m being 1 or 2.

16. Compound of the formula I according to claim 10, wherein R¹⁰ is hydrogen, N-acetyl, N-isopropyl-2-aminoethyl, N-isoprenyl-2-aminoethyl or N-acetyl, N-methyl-2-aminoethyl, especially wherein R¹⁰ is hydrogen or N-acetyl, N-methyl-2-aminoethyl.

17. Compound of the formula I according to claim 10, wherein it is selected from the group of compounds I-1 to I-3, wherein the compound of the formula I-1 is one, in which R³=R⁶=OCH₃, R⁷ and R⁸ form together a group O—CH₂—O and R¹⁰=N-acetyl, N-methyl-2-aminoethyl;
the compound of the formula I-2 is one, in which R³=OCH₃, R⁶=R⁸=R¹⁰=H, and R⁷=cinnamoyloxy;
the compound of the formula I-3 is one, in which R³=benzoyloxy, R⁶==R¹⁰=H, and R⁷=R⁸=OCH₃; and
the compound of the formula I-4 is one, in which R³=OCH₃, R⁶=R⁸=R¹⁰=H, and R⁷=(3,4,5-trimethoxy)benzoyloxy.

18. The compound of the formula I as defined in claim 10 as blood glucose controlling agent, as insulin sensitizer, as blood lipid lowering agent, as pancreatic β-cell function improver, as (non-autoimmune) diabetes type 2 preventing agent and/or as Syndrome X preventing agent.

19. A pharmaceutical composition containing at least one compound of the formula I as defined in claim 1 and a conventional pharmaceutical carrier.

20. The pharmaceutical composition according to claim 19, wherein the compound of the formula I is selected from the group consisting of compounds of the formula I-1 to I-4, especially the compounds of the formula I-1 to I-3.

21. A method for the treatment of a disorder connected to impaired glucose metabolism and impaired insulin action in mammals including humans, said method comprising administering an effective dose of a compound of the formula I as defined in claim 1 to mammals including humans which are in need thereof.

22. The method according to claim 21, wherein the disorder connected to impaired glucose metabolism and impaired insulin action in mammals including humans is (non-autoimmune) diabetes type 2.

23. The method according to claim 21, wherein the mammal is a human, a cat, a dog or a horse.

24. Compound of the formula I-1, I-2, I-3 and I-4 as defined in claim 3 for use as medicament.

25. Compound of the formula I as defined in claim 8 for use as medicament.
